(19) United States
(12) Patent Application Publication
(10) Pub. No.: US 2012/0110902 A1
(43) Pub. Date: May 10, 2012

(54) SULPHUR-COMPOUND ADSORBING AGENT FOR SOLVENT EXTRACTION OF COAL, AND A SULPHUR-COMPOUND ADSORPTION METHOD AND COAL REFINING METHOD EMPLOYING THE SAME

(75) Inventors: Kwang Bok Yi, Daejeon (KR); Jong Nam Kim, Daejeon (KR); Si Hyun Lee, Daejeon (KR); Chang Hyun Ko, Daejeon (KR); Hee Tae Beum, Daejeon (KR); In Baek Kim, Daejeon (KR)

(73) Assignee: KOREA INSTITUTE OF ENERGY RESEARCH, Daejeon (KR)

(21) Appl. No.: 13/384,230

(22) PCT Filed: Nov. 18, 2009
(86) PCT No.: PCT/KR09/06809

§ 371 Date: Jan. 14, 2012

(30) Foreign Application Priority Data
Jul. 16, 2009 (KR) 10-2009-0064902

(51) Int. Cl. C10L 5/00; C07D 207/46; C01F 5/14; C09K 3/00; C01B 31/08; C07C 7/12; C01F 5/02

(52) U.S. Cl. 44/624; 585/823; 548/550; 585/824; 423/635; 252/184; 423/445 R

(57) ABSTRACT

A sulfur compound adsorbent for solvent extraction of coal and methods using the same to adsorb sulfur compounds and refine coal are provided. The adsorbent for solvent extraction of coal serves to remove sulfur compounds from an organic solvent containing a coal's combustible component resulting from solvent extraction of low-grade coal and is composed of any one or a mixture of two or more selected from among alkali earth metal oxide, alkali earth metal hydroxide, aluminum oxide and activated carbon.

### TECHNICAL FIELD

The present invention relates to a sulfur compound adsorbent for solvent extraction of coal and methods of using the same to adsorb sulfur compounds and refine coal. More particularly, the present invention relates to a sulfur compound adsorbent for solvent extraction of coal, which is composed of any one or a mixture of two or more selected from among alkali earth metal oxide, alkali earth metal hydroxide, aluminum oxide and activated carbon, and to methods of using the same to adsorb sulfur compounds and refine coal.

### BACKGROUND ART

Coal emits more carbon dioxide per unit of energy than other energy sources when burnt, and thus is recognized as a major cause of environmental pollution and global warming. However, coal reserves are significantly more abundant and more widely and evenly dispersed than petroleum and is an important energy source for human consumption.

In order to reduce pollutant emission and keep the air clean, many countries are investing large amounts of money in the development of alternative energy. However, it is expected that a significant amount of time will be required before alternative energy is used as an actual energy source. Humans will rely on fossil fuels until sufficient energy sources capable of substituting for fossil fuels are developed.

Technology for minimizing pollution in the use of coal that forms a significant portion of fossil fuels is very important. In this effort, technology of producing ashless coal from low-grade coal has recently been studied.

The largest portion of pollutants which are contained in coal is ash accounting for about 10% of coal. Coal ash is deposited on the surface of water wall tubes at the top of burners in power generation plants to cause the slagging phenomenon which interferes with the transfer of heat of combustion and reduces heat efficiency.

Accordingly, removal of ash from coal can prevent the occurrence of such deposition and slagging phenomena to increase heat transfer efficiency, thus increasing power generation efficiency and reducing CO₂ emission. In other words, successfully produced ashless coal can be burned directly in gas turbines in power generation plants, thus increasing power generation efficiency and reducing the generation of CO₂ per unit of energy.

Accordingly, technologies for removing ash from coal by chemical pretreatment have been actively developed. Such chemical pretreatment technologies can be generally divided into a process of extracting ash using an acid or a salt, and a process of extracting only a combustible portion using an organic solvent while leaving a component having a high ash content.

The process of extracting ash using an acid or a salt has an advantage of high production efficiency, even though the refined product has an ash content of 0.1-0.7%. On the other hand, the process of extracting a combustible component using an organic solvent has a disadvantage of low production efficiency, even though the product has an ash content of about 200 ppm, and thus a high content of the combustible component.

In both the processes, the product includes not only ash containing trace amounts of alkali metal ions, but also sulfur compounds which are not removed in the extraction process. The remaining alkali metal ions react with the sulfur compounds during combustion to form highly corrosive compounds such as sodium sulfate, which cause the corrosion and scaling of the parts of combustion engines.

Thus, in order to burn coal powder directly in a gas turbine, alkali metal ions or sulfur compounds should necessarily be removed after a coal refining process. Herein, the process of removing impurities including sulfur compounds should be operated in addition to the coal refining process, and thus, if the impurity removal process differs from the coal extraction process employing an organic solvent with respect to process conditions, not only installation costs but also operating costs can be greatly increased.

Thus, the coal extraction process using an organic solvent should be operated at a high temperature of 200˜400° C. and a high pressure of 10 bar, and the produced extract and the solvent are separated with phase change in a flash column having low pressure. For this reason, the process of removing alkali metal ions or the process of removing sulfur compounds should be operated under the same conditions as the coal extraction process.

A general coal extraction process will now be described in further detail with reference to FIG. 1. Coal and a solvent are mixed with each other to make a slurry mixture from which water is then removed in a water remover and preheater 1. Then, the slurry mixture is sent to a solvent extraction reactor 2 in which an extraction reaction occurs.

After completion of the extraction reaction, the dissolved liquid phase is passed through a filter system 3 to remove fine particulate materials therefrom, and the liquid phase passed through the filter system is dried in an extract dryer 4. The slurry-state material precipitated in the extraction reactor 2 is transferred and dried in a residual coal dryer 5.

Herein, on the outside of each of the water remover and preheater 1, the extraction reactor 2, the filter system 3, the extract dryer 4 and the residual coal dryer 5, a heater 6 is disposed. The solvents evaporated in the extraction dryer 4 and the residual coal dryer 5 is condensed into a liquid phase by passage through a condenser and recovered into a solvent recovery tank 8.

Studies on the above-described coal refining process have recently been actively conducted, but studies on the process of removing small amounts of impurities from coal are still insufficient. Particularly, Korean Patent Registration No. 836708 discloses a process of removing alkali metal ions using an inorganic ion exchanger. However, a study on a method in which sulfur compounds can be removed in the coal extraction process (which uses an organic solvent) in order to prevent the formation of highly corrosive compounds such as sodium sulfate has not yet been reported.

In other words, the technology of removing sulfur compounds in an organic solvent containing the combustible component of coal has not yet been attempted, and the technology of selectively removing sulfur compounds under conditions of high temperature and high pressure has also been reported. Generally, a hydrodesulfurization process that is carried out using large amounts of hydrogen and a catalyst is commercially used in a crude oil refining process, but when it is applied to the coal extraction process that uses an organic solvent, a great amount of cost will be consumed because the process conditions of the hydrodesulfurization process differ from those of the coal extraction process.

Accordingly, the present inventors have developed a coal refining process which can not only prevent alkali metal ions remaining in coal from reacting with sulfur compounds to form highly corrosive compounds when burnt, but also remove sulfur compounds without needing to add an additional process to an existing coal extraction process, thereby completing the present invention.

### DISCLOSURE

### Technical Problem

It is an object of the present invention to provide a sulfur compound adsorbent which can remove sulfur compounds from an organic solvent containing the combustible component of coal even under conditions of high temperature and high pressure, which are used in the solvent extraction process, so as to be able to prevent alkali metal ions remaining in coal from reacting with sulfur compounds to form highly corrosive compounds when burnt, and methods of using the sulfur compound adsorbent to adsorb sulfur compounds in an organic solvent and to refine coal.

Another object of the present invention is to provide a sulfur compound adsorbent which enables a process of removing impurities including sulfur compounds to be operated under conditions similar to those used in a coal extraction process employing an organic solvent, so as to eliminate the need to install and operate a sulfur compound removal process separate from a coal refining process, thus greatly reducing installation costs and operating costs, and methods of using the sulfur compound adsorbent to adsorb sulfur compounds in an organic solvent and to refine coal.

Still another object of the present invention is to provide a sulfur compound adsorbent which has resistance to organic solvents such as 1-MN or NMP, selectively removes sulfur compounds at high temperature and high pressure and shows excellent desulfurization performance, and methods of using the sulfur compound adsorbent to adsorb sulfur compounds in an organic solvent and to refine coal.

Yet another object of the present invention is to provide a sulfur compound adsorption method and a coal refining method, which can treat an organic solvent, which contains a coal's combustible component dissolved therein, in a continuous process.

### Technical Solution

In order to accomplish the above objects, the present invention provides an adsorbent for solvent extraction of coal, which serves to remove sulfur compounds from an organic solvent containing a coal's combustible component resulting from solvent extraction of low-grade coal and is composed of any one or a mixture of two or more selected from among alkali earth metal oxide, alkali earth metal hydroxide, aluminum oxide and activated carbon.

The present invention also provides a sulfur compound-adsorbing method comprising removing sulfur compounds from an organic solvent containing a coal's combustible extract using an adsorbent composed of any one or a mixture of two or more selected from among alkali earth metal oxide, alkali earth metal hydroxide, aluminum oxide and activated carbon.

The present invention also provides a coal refining method comprising the steps of: i) extracting a combustible component from low-grade coal using an organic solvent; ii) adsorbing and removing sulfur compounds from the combustible component-containing organic solvent of step i) using an adsorbent composed of any one or a mixture of two or more selected from among alkali earth metal oxide, alkali earth metal hydroxide, aluminum oxide and activated carbon; and iii) separating the combustible extract from the sulfur compound-removed organic solvent of step ii).

Herein, said aluminum oxide or activated carbon is preferably impregnated with a transition metal, in which the transition metal is preferably nickel (Ni). Also, the content of the nickel (Ni) is preferably 1-15 wt %.

Also, said alkali earth metal oxide is preferably CaO or MgO, and said alkali earth metal hydroxide is preferably Ca(ON)₂ or Mg(OH)₂.

Also, adsorbing the sulfur compounds from the coal's combustible component-containing organic solvent is preferably carried out at a temperature of 200˜400° C. and a pressure of 5-15 bar.

In addition, the organic solvent is preferably 1-MN (1-methylnaphthalene) or NMP (N-methyl-2-pyrrolidone).

### Advantageous Effects

The sulfur compound adsorbent of the present invention and the methods of using the Sulfur compound adsorbent to adsorb sulfur compounds in an organic solvent and refine coal can efficiently and economically remove sulfur compounds from an organic solvent containing the combustible component of coal by using a simple reactor containing an adsorbent without additional energy in the coal solvent extraction process of removing high-concentration ash from low-grade coal and extracting only combustible component from the coal.

In other words, according to the present invention, sulfur compounds can be removed from an organic solvent containing the combustible component of coal under process conditions similar to those (such as high temperature and high pressure) used in the coal extraction process, and thus installation costs and operating costs can be greatly reduced and alkali metal ions remaining in coal can be prevented from reacting with sulfur compounds to form highly corrosive compounds when coal-powder is burned directly in a gas turbine.

Also, the sulfur compound adsorbent of the present invention has advantages in that it is composed of an inexpensive natural mineral or a widely used reforming catalyst such as activated carbon or Ni/Al₂O₃, has resistance to organic solvents such as 1-MN or NMP and can selectively remove sulfur compounds at high temperature and high pressure, thus exhibiting excellent desulfurization performance.

In addition, the inventive methods for adsorbing sulfur compounds and refining coal can treat an organic solvent containing the combustible component of coal in a continuous process, and thus reduce the content of sulfur compounds by 50% or more while minimizing additional costs in a process of producing ultra-clean coal.

### DESCRIPTION OF DRAWINGS

FIG. 1 is a view showing a process for solvent extraction of coal.

FIG. 2 shows the results of carrying out a test for batchwise removal of sulfur compounds from a 1-MN solution prepared by liquefying Drayton coal (extraction solvent: 1-MN, coal: Drayton, reaction temperature: 350° C., reaction pressure: 10 bar).

FIG. 3 shows the results of carrying out a test for batchwise removal of sulfur compounds from a 1-MN solution prepared by liquefying ROTO coal (extraction solvent: 1-MN, coal: Roto, temperature: 350° C., reaction pressure: 10 bar).

FIG. 4 is a view showing the configuration of an apparatus for continuous removal of sulfur compounds.

FIG. 5 shows the results of carrying out a test: for continuous removal of a sulfur compound using a simulated solution (extraction solvent: 1-MN, sulfur compound: Thiopheneo, temperature: 200˜350° C., reaction pressure: 5-20 bar).

FIG. 6 shows the results of carrying out a test for continuous removal of sulfur using a 1-MN solvent containing a combustible extract of coal (extraction solvent: 1-MN, coal: Drayton, temperature: 350° C., reaction pressure: 10 bar).

### DESCRIPTION OF THE REFERENCE NUMERALS IN THE DRAWINGS

1: water remover and preheater; 2: extraction reactor;

3: filter system; 4: extract dryer;

5: residual coal dryer; 6: heater;

7: condenser; 8: solvent recovery tank;

10: liquefied coal storage tank; 20: high-pressure liquid Pump;

30: adsorbent; 40: high-temperature furnace;

50: pressure gauge; 60: pressure control valve;

70: sample collection container.

### BEST MODE

Hereinafter, a sulfur compound adsorbent for solvent extraction of coal according to the present invention and methods of using the same to adsorb sulfur compounds and refine coal will be described in detail with reference to the accompanying drawings.

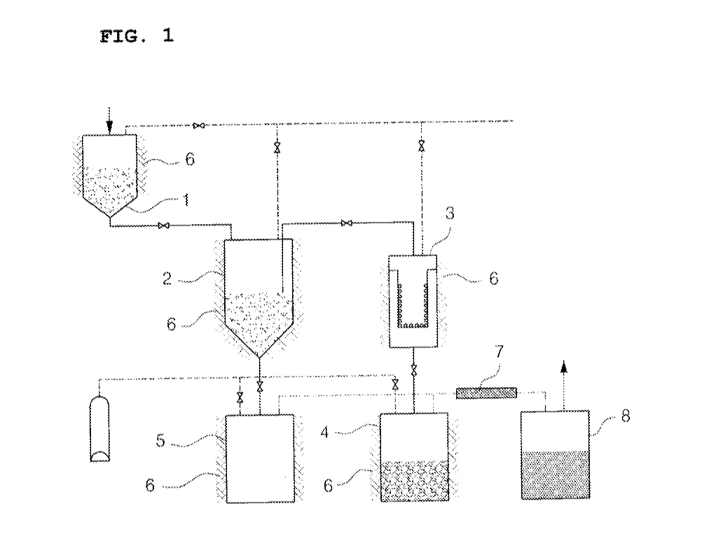

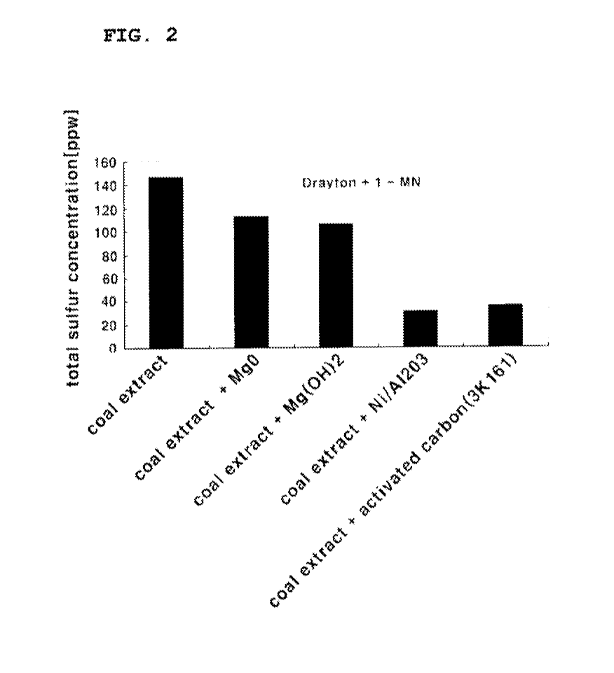

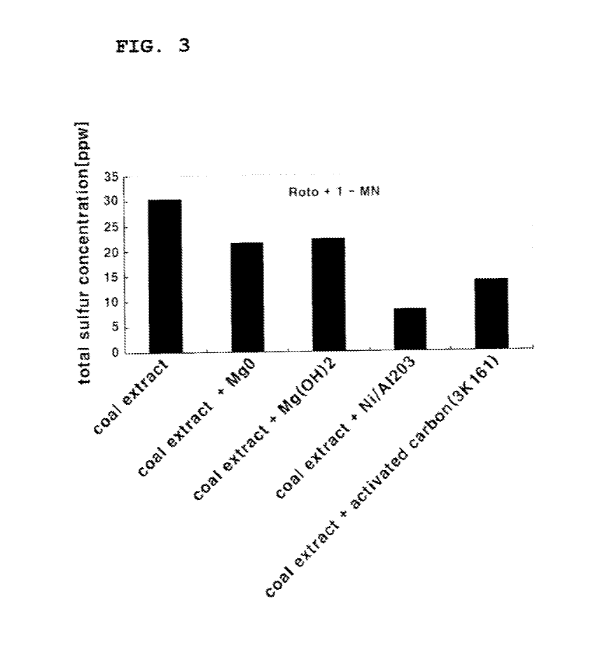

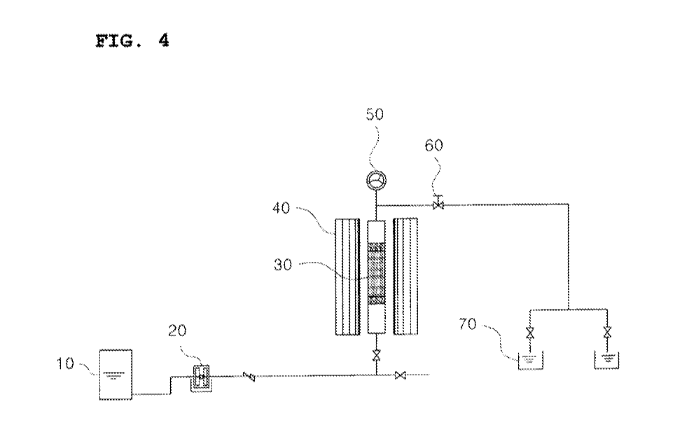

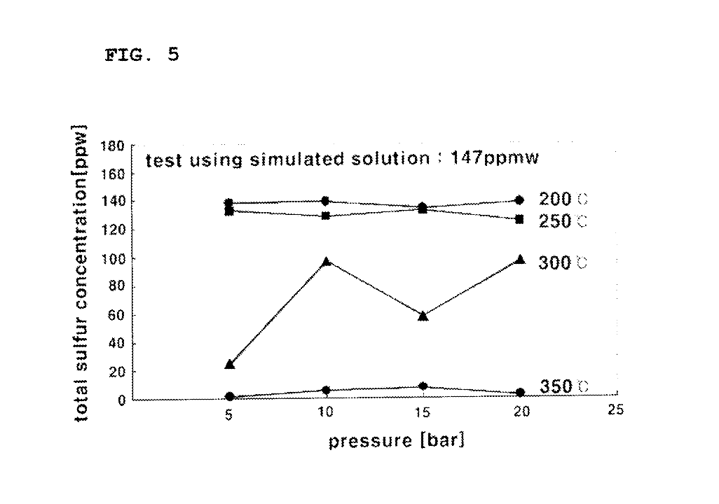

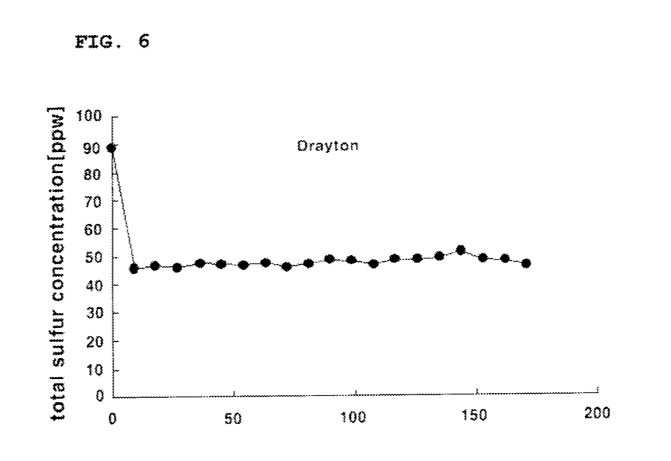

FIG. 1 is a view showing a general process for coal extraction of coal; FIG. 2 is a graphic diagram showing the results of carrying out a test for batchwise removal of sulfur compounds from a 1-MN solution prepared by liquefying Drayton coal; FIG. 3 is a graphic diagram showing the results of carrying out a test for batchwise removal of sulfur compounds from a 1-MN solution prepared by liquefying ROTO coal; FIG. 4 is a view showing the configuration of an apparatus for continuous removal of sulfur compounds; FIG. 5 is a graphic diagram showing the results of carrying out a test for continuous removal of sulfur using a simulated solution; and FIG. 6 is a graphic diagram showing the results of carrying out a test for continuous removal of sulfur using a 1-MN solvent containing a combustible extract of coal.

The sulfur compound adsorbent for solvent extraction of coal according to the present invention serves to remove sulfur compounds from an organic solvent containing the combustible component of coal resulting from solvent extraction of low-grade coal and is composed of any one or a mixture of two or more selected from alkali earth metal oxide, alkali earth metal hydroxide, aluminum oxide (Al₂O₃, CuCl/Al₂O₃) and activated carbon.

The adsorbent can remove several thousand to several thousand ppm of sulfur compounds from an organic solvent and can completely remove organic sulfur components such as thiophene and inorganic sulfur components such as FeS or FeS₂.

Herein, the aluminum oxide or the activated carbon is preferably impregnated with a transition metal, in which the transition metal is preferably nickel (Ni). Herein, the content of the nickel (Ni) is preferably in the range of 1 wt % to 15 wt %. Also, the alkali earth metal oxide may be CaO or MgO, and the alkali earth metal hydroxide may be Ca(OH)₂ or Mg(OH)₂.

The adsorbent of the present invention can adsorb sulfur compounds from an organic solvent containing the combustible extract of coal at a temperature of 200˜400° C. and a pressure of 5-15 bar.

In other words, the adsorbent of the present invention can remove sulfur compounds from an organic solvent containing the combustible component of coal under process conditions similar to those (such as high temperature and high pressure) used in the coal extraction process, and thus can greatly reduce installation costs and operating costs and can prevent alkali metal ions remaining in coal from reacting with sulfur compounds to form highly corrosive compounds when coal powder is burned directly in a gas turbine.

Also, the organic solvent that is used in the present invention may be any organic solvent capable of extracting a combustible component from coal. Preferably, the organic solvent may be 1-MN (1-methylnaphthalene) or NMP (N-methyl-2-pyrrolidone).

The sulfur compound adsorbent of the present invention is composed of an inexpensive natural mineral or a widely used reforming catalyst such as activated carbon or Ni/Al₂O₃, has resistance to organic solvents such as 1-MN or NMP and can selectively remove sulfur compounds at high temperature and high pressure, thus exhibiting excellent desulfurization performance.

Meanwhile, the sulfur compound-adsorbing method of the present invention comprises removing sulfur compounds from an organic solvent containing a combustible extract of coal using an adsorbent composed of any one or a mixture of two or more selected from alkali earth metal oxide, alkali earth metal hydroxide, aluminum oxide and activated carbon.

As described above, the aluminum oxide or activated carbon that is used as the adsorbent in the adsorbing method may be impregnated with a transition metal, in which the transition metal is preferably nickel (Ni). Herein, the content of the nickel (Ni) is preferably in the range of 1 wt % to 15 wt %. Also, the alkali earth metal oxide may be CaO or MgO, and the alkali earth metal hydroxide may be Ca(OH)₂ or Mg(OH)₂.

The above-described adsorbing method may be carried out using an organic solvent containing a combustible extract of coal at a temperature of 200-400° C. and a pressure of 5-15 bar. In other words, the adsorbing method of the present invention can remove sulfur compounds from an organic solvent containing a combustible extract of coal under process conditions similar to those (such as high temperature and high pressure) used in the coal extraction process, and thus can treat the organic solvent, which contains the coal's combustible component dissolved therein, in a continuous process. Accordingly, the adsorbing method of the present invention can reduce the content of sulfur compounds while minimizing additional costs in the process of producing ultra-clean coal.

Also, the organic solvent that is used in the adsorbing method of the present invention may be any solvent capable of extracting a combustible component from coal. Preferably, the organic solvent may be 1-MN (1-methylnaphthalene) or NMP (N-methyl-2-pyrrolidone).

Meanwhile, the coal refining method of the present invention comprises the steps of: i) extracting a combustible component from low-grade coal using an organic solvent; ii) adsorbing and removing sulfur compounds from the combustible extract-containing organic solvent of step i) using an adsorbent composed of any one or a mixture of two or more selected from alkali earth metal oxide, alkali earth metal hydroxide, aluminum oxide and activated carbon; and iii) separating the extract from the sulfur compound-removed organic solvent of step ii).

As described above, the aluminum oxide or activated carbon that is used as the adsorbent in the adsorbing step of step ii) may be impregnated with a transition metal, in which the transition metal is preferably nickel (Ni). Herein, the content of the nickel (Ni) is preferably in the range of 1 wt % to 15 wt %. Also, the alkali earth metal oxide that is used as the adsorbent in the adsorbing step of step ii) may be CaO or. MgO, and the alkali earth metal hydroxide may be Ca(OH)₂ or Mg(OH)₂.

The adsorbing step may be carried out in an organic solvent containing a combustible extract of coal at a temperature of 200˜400° C. and a pressure of 5-15 bar, which are similar to the conditions used in the coal extraction step of step i). The adsorbing step can efficiently remove sulfur compounds from the organic solvent containing the coal's combustible component dissolved therein.

Also, the organic solvent that is used in the adsorbing method of the present invention may be any organic solvent callable of a combustible component from coal. Preferably, the organic solvent may be 1-MN (1-methylnaphthalene) or NMP (N-methyl-2-pyrrolidone).

Hereinafter, examples for adsorbing sulfur compounds by batch-type and continuous type processes using the adsorbent of the present invention will be described.

An organic solvent containing a coal's combustible component, used in the following examples, was obtained by mixing low-grade coal (Drayton or Roto) having a high ash content with 1-MN (1-methyl naphthalene), allowing the mixture to react at 350° C. and 10 atm for 1 hour so as to extract a combustible component from the coal, and filtering the resulting material through a 2-μm ceramic filter. Also, a test for removal of sulfur compounds was carried out under the same pressure and temperature conditions as those used in the process of extracting the combustible component from coal.

Also, adsorbents such as Ca(OH)₂, CaO, MgO and Mg(OH)₂ were obtained by calcining samples (purchased from Aldrich) at a temperature of 350° C. or above in a nitrogen atmosphere, and Ni/Al₂O₃ was obtained by impregnating a Ni-containing precursor solution into Al₂O₃ and calcining the impregnated material in a nitrogen atmosphere. Also, activated carbon used as an adsorbent was obtained by treating commercial activated carbon at a temperature of 900° C. or above for 3 hours or more in a carbon dioxide atmosphere in order to increase the specific surface are of the activated carbon.

### EXAMPLE 1

### Batch-Type Test for Removal of Sulfur Compounds

For a batch-type test for removal of sulfur compounds, a stainless reactor having a volume of 10 ml was manufactured. Both ends of the reactor were mounted with spiral stoppers such that the reactor could be open and closed. 5 g of a 1-MN solution containing the coal's combustible component dissolved therein and 0.5 g of an adsorbent such as MgO, Mg(OH)₂, Ni/Al₂O₃ or activated carbon were introduced into the reactor which was then sealed and shaken well to mix the solution with the adsorbent.

Then, the reactor was placed in an open and close-type heating furnace, and the temperature of the reactor was increased to 370° C. for 2 hours, and then maintained at that temperature for 1 hour. While the temperature was maintained at 370° C., the reactor was rotated at 20-minute intervals such that the adsorbent could be sufficiently brought into contact with the 1-MN solution containing the coal's combustible component dissolved therein. Then, the reactor was cooled to room temperature, and the reaction product was taken out of the reactor and filtered through a 0.2-μm filter. Then, the total sulfur concentration of the resulting solution was measured by X-ray fluorescence analysis.

FIG. 2 shows the results of a batch type test for removal of sulfur compounds from a 1-MN solution prepared by liquefying Drayton coal. As can be seen in FIG. 2, the 1-MN solution contained 147 ppmw of sulfur before reaction with the adsorbent, but the sulfur concentration of the 1-MN solution decreased after reaction with the adsorbent. In the case of MgO or Mg(OH), about 25% of sulfur was removed, but in the case of Ni/Al₂O₃ and activated carbon, about 73% of sulfur was removed.

FIG. 3 shows the results of the same test as that of FIG. 2, carried out using ROTO coal. ROTO coal has a sulfur content lower than Drayton coal, and thus the prepared ROTO 1-MN solution also had a sulfur content lower than the Drayton 1-MN solution. As can be seen from the test results in FIG. 3, Ni/Al₂O₃ removed 73% or more of sulfur and showed the most excellent ability to remove sulfur, like the test results obtained using the Drayton coal.

### EXAMPLE 2

### Continuous Type Test for Removal of Sulfur Compounds Using Simulated Solution

Before a sulfur compound removal test was carried out using liquefied coal in a continuous process, a simulated solution was constructed and used in a continuous type test for removal of sulfur compounds.

FIG. 4 is a view showing the construction of the continuous process. As shown in FIG. 4, an organic solvent stored in a liquefied coal storage tank 10 is moved by a high-pressure liquid pump 20 into a reactor containing an adsorbent 30, and the reactor containing the adsorbent 30 is heated by a high-temperature 40. The pressure within the reactor is indicated in a pressure gauge 50 and controlled by a pressure control valve 60. Finally, the organic solvent after completion of the adsorption reaction is collected in a sample collection container 70.

While the test was carried out, the temperature within the reactor was changed to 200° C., 250° C., 300° C. and 350° C., and the pressure within the reactor was changed to 5, 10, 15 and 20 bar at each of the temperatures. The simulated solution was passed through the adsorbent layer at a flow rate of 0.3 ml/min.

The adsorbent used was Ni/Al₂O₃ (Ni content: 10 wt %), and quartz wool was placed over and under the adsorbent in the reactor such that the adsorbent could be fixed. The pressure within the reactor was controlled by the pressure control valve, and after completion of the reaction, the liquefied coal was placed in the sample collection container through two sample ports by operating the valve. The sulfur content of the liquefied material in the collection container was continuously measured by X-ray fluorescence analysis at 30-minute intervals after the start of the reaction.

The simulated solution was prepared by adding thiophene to 1-methyl naphthalene so as to reach a total sulfur concentration of 147 ppmw. The prepared simulated solution was brought into contact with the adsorbent under reaction conditions of high temperature and high pressure as described above, and then used in the analysis of total sulfur concentration.

FIG. 5 shows the results of the sulfur compound removal test carried out using the simulated solution. As can be seen in FIG. 5, the ability of the adsorbent to remove sulfur did not substantially change depending on pressure, but did greatly change depending on temperature. At 200° C. and 250° C., the adsorbent had little or no ability to remove sulfur, but at 300° C., the adsorbent showed the ability to remove 60% or more of sulfur at 5 bar. At 350+ C., the adsorbent showed the ability to remove 95% or more of sulfur at all the pressures.

### EXAMPLE 3

### Continuous Type Test for Removal of Sulfur Compounds Using 1-MN Solution Containing Coal's Combustible Component Dissolved Therein

Because Ni/Al₂O₃ had an excellent ability to remove sulfur from the stimulated solution, a continuous type test for sulfur removal was carried out using a 1-MN solution obtained by extracting Drayton coal. In the test, the continuous processing apparatus for sulfur removal shown in FIG. 4 was used, in which the 1-MN solution containing the coal's combustible extract was received in the storage tank and transferred by the high-pressure pump into the reactor placed in the high-temperature furnace.

While the test was carried out, the temperature within the reactor was maintained at 350° C. The 1-MN solution containing the coal's combustible extract was passed through the adsorbent layer at a flow rate of 0.3 ml/min. The adsorbent used was Ni/Al₂O₃ (Ni content: 10 wt %), and quartz wool was placed over and under the adsorbent in the reactor such that the adsorbent could be fixed.

The pressure within the reactor was controlled by the pressure control valve and maintained at 10 bar while the test was carried out. After completion of the reaction, the 1-MN solution was placed in the sample collection container through two sample ports by operating the valve. The sulfur content of the collected sample was continuously measured by X-ray fluorescence analysis at 30-minute intervals after the start of the reaction.

FIG. 6 shows the results of the continuous type test for sulfur removal, carried out using the 1-MN solution containing the coal's combustible extract. The x-axis of the graph in FIG. 6 denotes the volume of the 1-MN solution containing the coal's combustible extract, treated by 1 g of Ni/Al₂O₃, and the y-axis denotes the total sulfur concentration of the 1-MN solution containing the coal's combustible extract, measured after the sulfur removal test.

When 170 ml of the 1-MN solution was treated with 1 g of Ni/Al₂O₃, the total sulfur concentration of the 1-MN solution was decreased from 90 ppmw to 45 ppmw. Also, while 170 ml of the 1-MN solution was completely treated, the breakdown of the total sulfur concentration did not occur, suggesting that the adsorbent had an excellent ability to receive sulfur.

As can be seen in the above examples, when a simple small-sized reactor is installed in the process of extracting coal with an organic solvent, the concentration of sulfur in liquefied coal can be reduced by 50% or more without using additional energy.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An adsorbent for solvent extraction of coal, which serves to remove sulfur compounds from an organic solvent containing a coal's combustible component resulting from solvent extraction of low-grade coal and is composed of any one or a mixture of two or more selected from among alkali earth metal oxide, alkali earth metal hydroxide, aluminum oxide and activated carbon.

2. The adsorbent of claim 1, wherein the aluminum oxide or the activated carbon is impregnated with a transition metal.

3. The adsorbent of claim 2, wherein the transition metal is nickel (Ni).

4. The adsorbent of claim 3, wherein the content of the nickel (Ni) is 1-15 wt %.

5. The adsorbent of claim 1, wherein the alkali earth metal oxide is CaO or MgO.

6. The adsorbent of claim 1, wherein the alkali earth metal hydroxide is Ca(OH)₂ or Mg (OH)₂.

7. The adsorbent of claim 1, wherein adsorbing the sulfur compounds in the organic solvent containing the coal's combustible component is carried out at a temperature ranging from 200° C. to 400° C.

8. The adsorbent of claim 1, wherein adsorbing the sulfur compounds in the organic solvent containing the coal's combustible component is carried out is carried out at a pressure ranging from 5 bar to 15 bar.

9. The adsorbent of claim 1, wherein the organic solvent is 1-MN (1-methylnaphthalene) or NMP (N-methyl-2-pyrrolidone).

10. A sulfur compound-adsorbing method comprising removing sulfur compounds from an organic solvent containing a coal's combustible extract using an adsorbent composed of any one or a mixture of two or more selected from among alkali earth metal oxide, alkali earth metal hydroxide, aluminum oxide and activated carbon.

11. The sulfur compound-adsorbing method of claim 10, wherein the aluminum oxide or the activated carbon is impregnated with a transition metal.

12. The sulfur compound-adsorbing method of claim 11, wherein the transition metal is nickel (Ni).

13. The sulfur compound-adsorbing method of claim 12, wherein the content of the nickel (Ni) is 1-15 wt %.

14. The sulfur compound-adsorbing method of claim 10, wherein the alkali earth metal oxide is CaO or MgO.

15. The sulfur compound-adsorbing method of claim 10, wherein the alkali earth metal hydroxide is Ca(OH), or Mg(OH)₂.

16. The sulfur compound-adsorbing method of claim 10, wherein adsorbing the sulfur compounds in the organic solvent containing the coal's combustible component is carried out at a temperature ranging from 200° C. to 400° C.

17. The sulfur compound-adsorbing method of claim 10, wherein adsorbing the sulfur compounds in the organic solvent containing the coal's combustible component is carried out is carried out at a pressure ranging from 5 bar to 15 bar.

18. The sulfur compound-adsorbing method of claim 10, wherein the organic solvent is 1-MN (1-methylnaphthalene) or NMP (N-methyl-2-pyrrolidone).

19. A coal refining method comprising the steps of:
i) extracting a combustible component from low-grade coal using an organic solvent;
ii) adsorbing and removing sulfur compounds from the combustible extract-containing organic solvent of step i) using an adsorbent composed of any one or a mixture of two or more selected from among alkali earth metal oxide, alkali earth metal hydroxide, aluminum oxide and activated carbon; and
iii) separating the combustible extract from the sulfur compound-removed organic solvent of step ii).

20. The coal refining method of claim 19, wherein the aluminum oxide or the activated carbon is impregnated with a transition metal.

21. The coal refining method of claim 20, wherein the transition metal is nickel (Ni).

22. The coal refining method of claim 21, wherein the content of the nickel (Ni) is 1-15 wt %.

23. The coal refining method of claim 19, wherein the alkali earth metal oxide is CaO or MgO.

24. The coal refining method of claim 19, wherein the alkali earth metal hydroxide is Ca(OH)₂ or Mg(OH)₂.

25. The coal refining method of claim 19, wherein adsorbing the sulfur compounds in the organic solvent containing the coal's combustible component is carried out at a temperature ranging from 200° C. to 400° C.

26. The coal refining method of claim 19, wherein adsorbing the sulfur compounds in the organic solvent containing the coal's combustible component is carried out is carried out at a pressure ranging from 5 bar to 15 bar.

27. The coal refining method of claim 19, wherein the organic solvent is 1-MN (1-methylnaphthalene) or NMP (N-methyl-2-pyrrolidone).
